(19) United States
(12) Patent Application Publication
(10) Pub. No.: US 2006/0234359 A1
(43) Pub. Date: Oct. 19, 2006

(54) Method for the production or r-a-lipoic acid by fermentation

(76) Inventor: Tobias Dassler, Munich (DE)

Correspondence Address: WILLIAM COLLARD;COLLARD & ROE, P.C., 1077 NORTHERN BOULEVARD, ROSLYN, NY, 11576, US

(21) Appl. No.: 10/538,157

(22) PCT Filed: Dec. 4, 2003
(86) PCT No.: PCT/EP03/13728

§ 371 Date: Jun. 8, 2005

(30) Foreign Application Priority Data
Dec. 12, 2002 (DE) 102 58 127.4

(51) Int. Cl. C12P 17/00; C07D 339/02

(52) U.S. Cl. 435/117; 549/39

(57) ABSTRACT

The invention relates to a method for the production of R-α-lipoic acid by fermentation, characterised in that a cell with a weakened lipoyl protein ligase A activity is cultivated in a culture medium, whereby the cell precipitates enantiomerically pure R-α-lipoic acid in the free form in the culture medium and the enantiomerically pure R-α-lipoic acid is separated off from the culture medium.

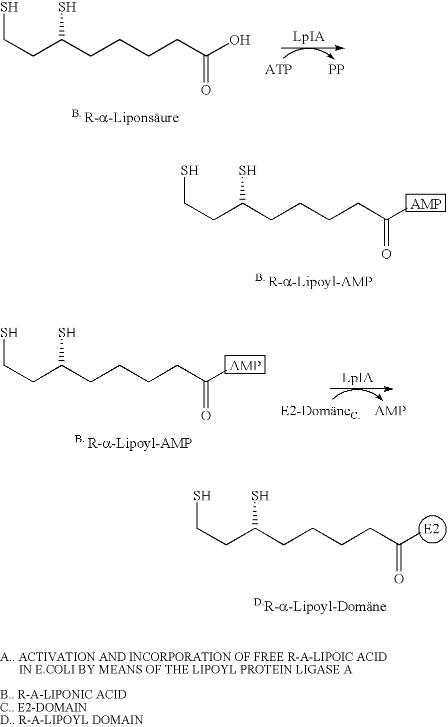

The invention relates to a method for fermentative production of R-α-lipoic acid and to cells which are particularly suitable for said method.

R-α-lipoic acid is an essential cofactor of particular multienzyme complexes in a multiplicity of pro- and eukaryotes. R-α-lipoic acid is bound in each case covalently via its carboxyl group to the ε-amino group of a specific lysine residue of the respective enzyme with the formation of a “lipoamide”. In this way, R-α-lipoic acid is part of the E2 subunit of pyruvate dehydrogenase (PDH) [EC 2.3.1.12] and of α-ketoglutarate dehydrogenase (KGDH) [EC 2.3.1.61] and plays an important part there as redox partner and acyl group donor in oxidative decarboxylation of α-keto acids. Moreover, R-α-lipoic acid acts as aminomethyl carrier in glycine cleavage enzyme systems.

α-Lipoic acid is an optically active molecule having a center of chirality on the C6 carbon atom. The R configuration of α-lipoic acid is the naturally occurring enantiomer. Only this form is physiologically active as cofactor of the corresponding enzymes. α-Lipoic acid may occur both in an oxidized (5-[1,2]-dithiolan-3-yl-pentanoic acid) and in a reduced form (6,8-dimercaptooctanoic acid). The term “α-lipoic acid” means hereinbelow both forms and the particular salts of α-lipoic acid, such as, for example, the calcium, potassium, magnesium, sodium or ammonium salt.

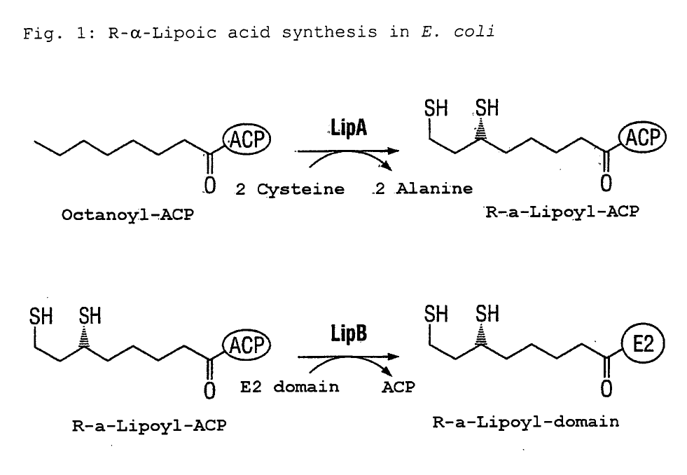

The biosynthesis of R-α-lipoic acid has been studied particularly intensively on the bacterium Escherichia coli (see FIG. 1). Here, octanoic acid which is covalently bound to the acyl-carrier protein (ACP) serves as specific precursor in the lipoic acid synthesis. In a complex reaction, two sulfur atoms are transferred to the thus activated octanoic acid (octanoyl-ACP), giving R-α-lipoyl-ACP. This reaction is catalyzed by lipoic acid synthase [EC 2.8.1.-], the lipA gene product. Serving as sulfur donor is ultimately the amino acid L-cysteine. Subsequent transfer of R-α-lipoic acid from R-α-lipoyl-ACP to the E2 subunit of the α-keto acid dehydrogenases is catalyzed by lipoyl-protein ligase B [EC 6.-.-.-], the lipB gene product, without, however, R-α-lipoyl-ACP or R-α-lipoic acid appearing as free intermediates (Miller et al., 2000, Biochemistry 39:15166-15178).

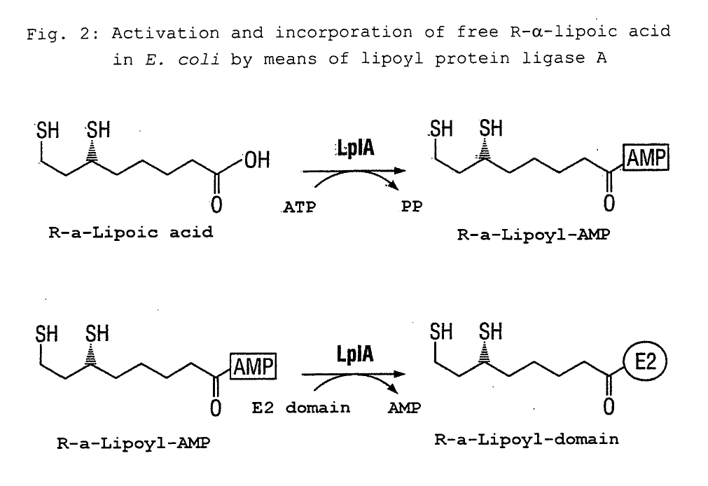

However, E. coli can also take up free R-α-lipoic acid from the surrounding medium and use it for the generation of functional α-keto acid dehydrogenases. For this purpose, R-α-lipoic acid is first activated by means of ATP to R-α-lipoyl-AMP and then transferred to the corresponding enzyme subunits (see FIG. 2). Both activities are catalyzed by lipoyl protein ligase A [EC 6.-.-.-], the lplA gene product (Morris et al., 1994, J. Biol. Chem. 269: 16091-16100). This LplA activity, however, is non-essential for E. coli wild-type strains, if the endogenous lipoic acid synthesis and the lipoyl group transfer is carried out via the LipA/LipB pathway. Thus, for example, lplA mutants have been described which have no longer any detectable lipoyl protein ligase A activity and whose phenotype is not distinguishable from a wild-type cell under normal growth conditions (Morris et al., 1994, J. Biol. Chem. 269: 16091-16100; Morris et al., 1995, J. Bacteriol. 177: 1-10).

Little is known about R-α-lipoic acid biosynthesis in eukaryotes. It is assumed, however, that R-α-lipoic acid synthesis and transfer to the corresponding enzymes take place in the mitochondria of eukaryotic cells in a manner similar to that in bacteria.

Apart from its relevance as essential component of enzymes having a central role in metabolism, the importance of α-lipoic acid to-pharmacotherapy and as a food supplement (nutraceutical) was recognized already early on: owing to its two thiol groups, α-lipoic acid has a distinctive antioxidative activity and can thus protect the organism against harmful processes induced by oxidative stress. Moreover, α-dihydrolipoic acid, the reduced form of α-lipoic acid, is capable of regenerating directly or indirectly other oxidized natural antioxidants in the body, such as ascorbic acid or α-tocopherol, or also, in the case of a lack thereof, of replacing said antioxidants, owing to its property as a strong reducing agent. Accordingly, α-lipoic acid is of central importance in acting together with ascorbic acid, α-tocopherol and glutathione, the “network of antioxidants”. α-Lipoic acid is also employed in the prevention and control of type II diabetes mellitus and the damaging secondary effects thereof such as, for example, polyneuropathy, cataract or cardiovascular conditions.

Currently, the different biological activity of the two α-lipoic acid enantiomers is the subject of intensive studies, although there is more and more evidence coming to light of application of the pure R enantiomer of α-lipoic acid having distinct advantages, compared to the S form. Thus, it was shown in an in vitro experiment that only the natural R-α-lipoic acid leads to the formation of functional α-keto acid dehydrogenases. In contrast, the S enantiomer even had an inhibiting effect on stimulation of the enzyme activity by R-α-lipoic acid. The reduction of α-lipoic acid and thus regeneration of the antioxidatively active α-dihydrolipoic acid in the mitochondria are thus of essential importance to the cell. The activity of mammalian mitochondrial NADH-dependent lipoamide reductase is almost 20 times higher in combination with the R enantiomer than with the S form. In addition, R-α-lipoic acid has, compared to the S enantiomer, a distinctly stronger action on insulin-mediated glucose uptake and glucose metabolism of skeletal muscle cells of insulin-resistant rats. Moreover, the R form exhibited in an animal experiment antiphlogistic action, while the S form had rather an analgetic action. In order to avoid undesired side effects, it is therefore extremely desirable to administer α-lipoic acid in each case only in the enantiomerically pure form. Currently, industrial production of α-lipoic acid is carried out exclusively by means of chemical methods, with the final product formed always being the racemate of R form and S form (Yadav et al., 1990, J. Sci. Ind. Res. 49: 400-409). To obtain enantiomerically pure R-α-lipoic acid, various methods have been developed. It is possible, for example, to resolve the racemate of α-lipoic acid or of one of the synthesis intermediates either chemically by means of chiral auxiliaries (Walton et. al, 1954, J. Amer. Chem. Soc. 76: 4748; DE 4137773) or enzymically (Adger et al., 1995, J. Chem. Soc., Chem. Commun.: 1563-1564). In other methods, the formation of a racemate is prevented owing to an enantioselective synthesis step, it being possible to introduce the new center of chirality either chemically (DE 3629116; DE 19533881; Bringmann et al., 1999, Z. Naturforsch. 54b: 655-661; DE 10036516) or by stereospecific biotransformation by means of microorganisms (Gopalan and Jacobs, 1989, Tetrahedron Lett. 30: 5705-5708; Dasaradhi et al., 1990, J. Chem. Soc., Chem. Commun.: 729-730; DE 10056025). Other processes, in turn, start chemical synthesis of enantiomerically pure α-lipoic acid by using a naturally occurring chiral reactant such as, for example, S-maleic acid or D-mannitol (Brookes and Golding, 1988, J. Chem. Soc. Perkin Trans. I: 9-12; Rama Rao et al., 1987, Tetrahedron Lett. 28, 2183-2186). Due to partly complicated synthesis steps, low yields and high material costs, all known methods for producing enantiomerically pure R-α-lipoic acids are currently not economical.

These days, many low molecular weight natural substances such as, for example, antibiotics, vitamins or amino acids, are frequently produced industrially by means of a fermentative method using various strains of microorganisms.

The applications to the Deutsches Patent- und Markenamt with file numbers 10235270.4 and 10245993.2 describe a method in which enantiomerically pure R-α-lipoic acid is produced exclusively in a fermentation process, using cells which overexpress a lipoic acid-synthase gene and a lipoyl protein ligase B gene individually or else in combination. However, enantiomerically pure R-α-lipoic acid is produced to a still very limited extent so that these fermentative methods currently cannot yet compete with chemical synthesis.

Only in rare cases, however, does a single genetic manipulation in the course of the “metabolic engineering” of a wild-type strain result in sufficiently high overproduction of the desired compound. Rather, this requires a combination of targeted genetic manipulations which are frequently also supplemented by classical mutagenesis/screening approaches.

Accordingly, it is the object of the present invention to provide a more productive method for fermentative production of enantiomerically pure R-α-lipoic acid.

This object is achieved by a method for preparing enantiomerically pure R-α-lipoic acid, which is characterized in that a cell having an attenuated lipoyl protein ligase A activity is cultured in a culture medium, said cell secreting enantiomerically pure R-α-lipoic acid in free form into said culture medium and said enantiomerically pure R-α-lipoic acid being removed from said culture medium.

An attenuated lipoyl protein ligase A activity preferably means, in accordance with the present invention, that the intracellular activity of the LplA protein in the cell is reduced by 25 to 100%, particularly preferably by 75 to 100%, compared to a wild-type cell. Very particular preference is given to the intracellular activity of the LplA protein being completely eliminated.

Physiological and biochemical data indicate that lipoic acid is present in wild-type cells virtually always in bound form, since R-α-lipoic acid is already synthesized in an entirely protein-bound manner (cf. FIG. 1) (Herbert and Guest, 1975, Arch. Microbiol. 106: 259-266; Miller et al., 2000, Biochemistry 39:15166-15178). Lipoyl protein ligase A is not involved in the de novo synthesis of R-α-lipoic acid; rather, the activity of this enzyme comprises coupling free R-α-lipoic acid to the E2 subunits of α-keto acid dehydrogenases. Surprisingly, it was found now that a reduction in or the complete elimination of lipoyl protein ligase A activity in a wild-type strain results in the accumulation of free, enantiomerically pure R-α-lipoic acid in the culture medium of said cells, although all lipoyl-binding sites of the E2 subunits are saturated with R-α-lipoic acid, both in E. coli wild-type strain and in an lplA mutant (Packman et al., 1991, Biochem. J. 277: 153-158; Morris et al., 1995, J. Bacteriol. 177: 1-10), which thus lack the substrate of the LplA protein (an unloaded E2 subunit). In addition, expression of the lplA gene in an E. coli wild-type strain is, in any case, only extremely weak. Accordingly, only a few molecules (<10) of lipoyl protein ligase A are present in a cell (Green et al., 1995, Biochem. J. 309: 853-862). It is therefore all the more surprising that a reduction in or complete elimination of lipoyl protein ligase A activity results in the secretion of R-α-lipoic acid.

The secretion of free R-α-lipoic acid from the cells allows the product to be readily isolated from the culture medium after removing the biomass, without the need for disrupting the cells beforehand or the need for removing R-α-lipoic acid from the carrier protein bound thereto (ACP or the E2 subunit of α-keto acid dehydrogenases) by a complicated hydrolysis step involving heavy losses.

The lplA gene-encoded lipoyl protein ligase A activity means the lipoyl protein ligase activity of a cell, which has a distinct substrate preference for free R-α-lipoic acid compared to R-α-lipoyl-ACP. The LplA protein is about 100 times more active with free R-α-lipoic acid than with R-α-lipoyl-ACP. This clearly distinguishes the lipoyl protein ligase A activity of a cell from the lipoyl protein ligase B activity, which prefers R-α-lipoyl-ACP to free R-α-lipoic acid as substrate (see FIGS. 1 and 2).

The lipoyl protein ligase A gene is preferably a gene having the sequence SEQ ID NO: 1 or a functional variant of said gene.

A functional variant in accordance with the present invention is a DNA sequence which is derived from the sequence depicted in SEQ ID NO: 1 by deletion, insertion or substitution of nucleotides, with the enzymic activity and specificity of the lipoyl protein ligase A encoded by the gene being retained.

The lipoyl protein ligase A gene codes for a protein comprising the Sequence ID NO: 2 or for functional variants having a sequence homology to SEQ ID NO: 2 of more than 35%.

The sequence homology to SEQ ID NO: 2 is preferably more than 60%, and is particularly preferably more than 80%.

In the present invention, all of the homology values mentioned refer to results obtained using the GAP algorithm (GCG Wisconsin Package, Genetics Computer Group (GCG) Madison, Wis.).

A number of possibilities for attenuating an enzyme activity in a cell are known to the skilled worker. An attenuation may be achieved, for example, by reducing expression of the corresponding gene or by replacing the chromosomal wild-type gene with a mutated allele which codes for an enzyme with reduced activity. In an extreme case, the enzyme activity may also be completely eliminated.

Expression of a gene may be reduced or prevented, for example, by the following measures:
attenuating the promoter by suitable base substitutions
inactivating/modifying a transcription activator required for expression
attenuating translation start signals (e.g. ribosomal binding site, start codon) by suitable base substitutions
removing mRNA-stabilizing regions of the gene
overexpressing of DNA regions coding for specific antisense RNA
deleting the entire gene or at least a crucial part thereof
destroying the gene by inserting an antibiotic resistance cassette, for example.

Mutated alleles of a gene which code for an enzyme with reduced activity may be generated, for example, by the following measures:
introducing reading-frame shifts to the corresponding gene due to nucleotide deletions or insertions
introducing specific base substitutions in the gene which result in the replacement of conserved amino acids or amino acids essential for activity.

Mutated alleles of the lplA gene may be generated using molecular-biological standard methods. A preferred possibility here is the introduction of specific base substitutions into the gene. This may be carried out, for example, by specifically altering the base sequence of the gene or of its promoter at one or more positions by using specific mutagenic primers during amplification of the lplA gene by means of polymerase chain reaction (PCR) (site-specific mutagenesis).

Particular preference is given to introducing a deletion into the lplA gene. This may be achieved by first cloning the gene, after amplification by means of PCR using specific primers which cover the complete lplA gene, into a plasmid vector (e.g. pUC18, pBR322, pACYC184). Restriction of the plasmid thus obtained with suitable restriction endonucleases which cut only in the lplA gene region makes it possible to remove internal regions of the gene. In this way, it is possible to introduce an internal deletion into the lplA gene, after religation of the restricted plasmid. As an alternative to religating the plasmid restricted in the lplA gene, it is also possible to clone an antibiotic resistance cassette into the lplA gene.

Methods for replacing any chromosomal DNA sequence with a sequence which, although homologous, has been altered by base insertions, base deletions or base substitutions are known to the skilled worker. Thus it is possible to use, for example, in Escherichia coli the system described by Link et al. (1997, J. Bacteriol. 179: 6228-6237), in order to replace the chromosomal wild-type sequence of the lplA gene with a mutated lplA allele via the mechanism of homologous recombination by means of integrative plasmids.

A preferred embodiment of the present invention uses cells which secrete enantiomerically pure R-α-lipoic acid into a culture medium and which have an attenuated lipoyl protein ligase A activity, said cells having, instead of a wild-type lplA gene, an lplA allele which has, in the base pair range 367-465, a base substitution which results in the LplA protein activity being reduced by at least 50%, or having a deletion in the lplA gene.

The present invention therefore also relates to a cell having the abovementioned properties.

The LplA protein activity is preferably reduced by from 50 to 100%, particularly preferably by from 75% to 100%.

In a particularly preferred embodiment of the cells of the invention, a base substitution in the gene region mentioned results in any LplA protein activity no longer being detectable.

In a very particularly preferred embodiment of the cells of the invention, the chromosome of the host organism contains merely an lplA gene fragment generated by an internal deletion, which can no longer code for a fractional lipoyl protein ligase A activity.

Cells having an attenuated lipoyl protein ligase A activity can be prepared by introducing into a starting cell an lplA allele coding for an lplA protein with an activity reduced by at least 50% compared to the wild-type protein instead of the lplA wild-type gene.

It was possible to identify genes coding for a lipoyl protein ligase A and genes required for de novo synthesis of R-α-lipoic acid (e.g. lipA, lipB) in a multiplicity of pro- and eukaryotic cells or organisms. It is therefore possible to prepare cells of the invention preferably from cells of pro- or eukaryotic organisms, which are capable of synthesizing R-α-lipoic acid themselves (starting cells), which are accessible to recombinant methods and which can be cultured by fermentation. Plant or animal cells which can be grown in cell culture are thus also suitable for preparing cells of the invention.

Cells of the invention may be prepared by using starting cells which have not been subjected to any manipulation previously.

Furthermore, however, it is possible to combine the cells of the invention also with measures which have already led to an improved production of R-α-lipoic acid. Particularly suitable are thus, for example, those cells which have already an increased lipoic acid synthase activity compared to the wild type, due to enhanced expression of the lipA gene compared to the wild type, and/or which have already an increased lipoyl protein ligase B activity compared to the wild type, due to enhanced expression of the lipB gene compared to the wild type. The patent applications DE 10235270 and DE 10245993 describe the preparation of cells having enhanced lipoic acid synthase activity compared to the wild type and/or enhanced lipoyl protein ligase B activity compared to the wild type.

The invention thus relates in particular also to cells which have, in addition to the at least 50% reduced or absent LplA protein activity, due to enhanced expression of the lipA gene, an increased lipoic acid synthase activity or which already have, due to enhanced expression of the lipb gene, an increased lipoyl protein ligase B activity.

The cells are preferably microorganisms such as, for example, yeast or bacterial strains. Said bacterial strains are particularly preferably of the family Enterobacteriaceae, very particularly preferably strains of the species Escherichia coli.

R-α-Lipoic acid can be recovered from the culture medium by methods known to the skilled worker, such as, for example, centrifugation of the cell-containing culture medium to remove the cells and by subsequent extraction and/or precipitation of the product.

The inventive cells for producing R-α-lipoic-acid are preferably cultured in a minimal salt medium known from the literature (Herbert and Guest, 1970, Meth. Enzymol. 18A, 269-272).

Carbon sources which may be used are in principle all usable sugars, sugar alcohols or organic acids or salts thereof. Preference is given to using aspartic acid, maleic acid, succinic acid, pyruvic acid, fumaric acid, glutamic acid, glucose, glycerol or oxaloacetic acid. Particular preference is given to succinic acid and oxaloacetic acid. Combined feeding with a plurality of different carbon sources is also possible. Furthermore, it is possible to add to the medium short-chain fatty acids having a chain length of C2-C8, preferably having a chain length of C6-C8 (hexanoic and octanoic acid, respectively), as specific precursors for the α-lipoic acid synthesis. The concentration of the carbon source added is preferably 0.1-30 g/l.

The cells of the invention are preferably incubated under aerobic culturing conditions and within the range of the optimum growth temperature for the particular cells over a period of 16-150 h.

Preference is given to an optimal temperature range of 15-55° C. Particular preference is given to a temperature between 30 and 37° C.

R-α-Lipoic acid produced in the method of the invention is detected and quantified, for example, by means of a bioassay using an indicator strain auxotrophic for lipoic acid (lipA mutant). This type of turbidimetric quantification of R-α-lipoic acid is known from the literature (Herbert and Guest, 1970, Meth. Enzymol. 18A, 269-272). The indicator strain used within the framework of the present invention, W1485lip2 (ATCC 25645), however, would also grow without supplemented R-α-lipoic acid, if the medium also contained acetate and succinate in addition to glucose. In order to prevent false-positive growth of said indicator strain in the bioassay when determining the R-α-lipoic acid produced, which growth is due, for example, to introduction of glucose and of acetic and succinic acid secreted by the producer strain in addition to R-α-lipoic acid, even the R-α-lipoic acid producer is preferably grown with succinate as sole carbon source. Said strain is supplemented with the supernatant of a cell culture of the invention; it is then possible to determine the lipoic acid content of the culture medium on the basis of indicator strain growth.

The examples below serve to further illustrate the invention. The bacterial strain Escherichia coli W3110ΔlplA used for carrying out said examples have been deposited according to the Budapest Treaty with the DSMZ (Deutsche Sammlung für Mikroorganismen und Zellkulturen GmbH, D-38142 Braunschweig, Germany) under the number DSM 15299. The plasmids pKP477 and pBAD-lipB are described in the patent application DE 10245993.

### EXAMPLE 1

### Construction of a Chromosomal Mutation in the lplA gene of the Host Organism

A) lplA Gene Amplification

The E. coli lplA gene was amplified by means of the polymerized chain reaction (PCR) using the Pwo DNA polymerase according to common practice known to the skilled worker. The template used was the chromosomal DNA of E. coli W3110 (ATCC 27325) wild-type strain. The primers used were the 3′-phosphorothioate-protected oligonucleotides lplA-fwd and lplA-rev having the following sequences:

1p1A-fwd:

(SEQ ID NO: 3)

5′-CGG CAT CCC TAT CTG CGC CTG ACA CTC GAC-3′
BamHI
1p1A-rev:

(SEQ ID NO: 4)

5′-CGG GAT CCT TTA TCT GAA CCG CCA TTT GCG CTG-3′
BamHI

The approx. 1.6 kb DNA fragment obtained in the PCR was then purified by means of a DNA adsorption column of the QIAprep spin miniprep kit (Qiagen, Hilden, Germany) according to the manufacturer's instructions.

B) Construction of Plasmid pKO3-ΔlplA

Cleavage sites for BamHI restriction endonuclease (recognition sequence underlined in the oligonucleotides) were introduced into the PCR fragment. The purified PCR fragment was cleaved by BamHI restriction endonuclease under the conditions indicated by the manufacturer, subsequently fractionated on an agarose gel and then isolated from said agarose gel by means of the GENECLEAN kit (BIO 101 Inc., La Jolla, Calif.) according to the manufacturer's instructions. The lplA gene was cloned by cleaving the pUC18 vector (Amersham Biosciences GmbH, Freiburg, Germany) by the BamHI restriction enzyme under the conditions indicated by the manufacturer, subsequently dephosphorylating the 5′ ends by treatment with alkaline phosphatase and then purifying said vector like the PCR fragment by means of the GENECLEAN method. The PCR fragment was ligated with a decreased and phosphorylated vector according to the manufacturer's instructions, using T4-DNA ligase. E. coli cells of the DH5a strain were transformed-with the ligation mixture by means of electroporation in a manner known to the skilled worker. The transformation mixture was applied to LB-ampicillin agar plates (10 g/l tryptone, 5 g/l yeast extract, 10 g/l NaCl, 15 g/l agar, 100 mg/l ampicillin) and incubated at 37° C. overnight.

The desired transformants were identified by restriction analysis, after isolating the plasmid by means of a QIAprep spin miniprep kit (Qiagen, Hilden, Germany). The plasmid obtained in this way is referred to as pUC18-lplA.

In order to introduce an internal deletion in the lplA gene, the pUC18-lplA vector was digested by restriction enzymes NruI and StuI which cut in each case once within the lplA gene, and the vector was religated as described above by means of T4-DNA ligase, then transformed and checked. As a result, 197 base pairs of a central region of the lplA gene were deleted and, at the same time, a frame shift was introduced, thereby inactivating the gene. The resulting plasmid, pUC18-ΔlplA, which now contains the truncated reading frame “ΔlplA” was cut by the enzyme BamHI, and the 1.4 kb DNA fragment which includes the ΔlplA gene fragment was cloned into the pKO3 vector (Link et al., 1997, J. Bacteriol. 179: 6228-6237) likewise cleaved by BamHI. The plasmid obtained in this way is referred to as pKO3-ΔlplA.

C) Replacement of the Chromosomal lplA Wild-Type Gene with the Deleted lplA allele from pKO3-ΔlplA

The plasmid pKO3-ΔlplA was introduced as described above into the W3110 strain by means of transformation, and plasmid-carrying clones were selected via the chloramphenicol resistance thus obtained (20 mg/l chloramphenicol). The chromosomal lplA wild-type gene was replaced with the deleted ΔlplA allele from pKO3-ΔlplA by means of homologous recombination according to the procedure of Link et al. (1997, J. Bacteriol. 179: 6228-6237), and plating of the cells on LB-sucrose agar plates made it possible to select simultaneously for dissolution of the cointegrates and for the loss of the plasmid which now contained the lplA wild-type gene. Sucrose-resistant single colonies were checked by means of PCR using the oligonucleotides lplA-fwd (SEQ ID NO: 3) and lplA-rev (SEQ ID NO: 4) with respect to whether the chromosomal replacement of the lplA wild-type gene with the deleted variant ΔlplA was successful. The strain generated in this way is referred to as W3110ΔlplA.

### EXAMPLE 2

### Preparation of R-α-lipoic Acid Producers

The lipB overexpression plasmid pBAD-lipB was transformed into the E. coli strains W3110ΔlplA and W3110 by means of electroporation and, after selection on LB agar plates containing 100 mg/l ampicillin, the plasmid was reisolated in each case from one of the transformants, cleaved with restriction endonucleases and checked. The control plasmid, pKP477, which contains, apart from the ampicillin resistance gene, only the regulatory sequences of the E. coli arabinose operons (araC-gene, araBAD-promoter region) was treated in a similar manner.

### EXAMPLE 3

### Fermentative Production of R-α-lipoic Acid

The strains mentioned in example 2, both with and without plasmid, were used for fermentative production of R-α-lipoic acid. As preculture for producer cultivation, 5 ml of LB liquid medium containing 100 mg/l ampicillin were firstly inoculated with the respective strain and incubated at 37° C. and 160 rpm on a shaker for 16 h. The cells were then harvested by centrifugation and washed twice with the corresponding volume of sterile saline (0.9% NaCl). The cells prepared in this way were finally used to inoculate 15 ml of BS medium (7 g/l K₂HPO₄; 3 g/l KH2PO₄; 1 g/l (NH₄)₂SO₄; 0.1 g/l MgSO₄×7 H₂O; 0.5 g/l Na₃ citrate×3 H₂O; 0.2% acid-hydrolyzed casein (vitamin-free); 13.5 g/l Na₂ succinate×6 H₂O; pH adjusted to 6.8 with HCl), which additionally contains 100 mg/l ampicillin, in a ratio 1:100. The producer cultures were incubated on a shaker at 37° C. and 160 rpm. Expression of the lipoyl protein ligase B gene on plasmid pBAD-lipB was induced by adding 0.2 g/l L-arabinose after approximately 4 h of incubation. After 24 h of incubation, samples were taken and the cells were removed from the culture medium by centrifugation. The R-α-lipoic acid contained therein was quantified by means of the known turbidimetric bioassay (Herbert and Guest, 1970, Meth. Enzymol. 18A: 269-272). Table 1 shows the free R-α-lipoic acid contents achieved in the particular culture supernatant after 24 h of incubation:

TABLE 1

| | R-α-lipoic acid |
|---|---|
| Strain | [μg/l] |
| W3110 | 0 |
| W3110ΔlplA | 25 |
| W3110 pKP477 | 0 |
| W3110ΔlplA pKP477 | 27 |
| W3110 pBAD-lipB | 25 |
| W3110ΔlplA pBAD-lipB | 191 |

1. A method for preparing enantiomerically pure R-α-lipoic acid, wherein a cell having an attenuated lipoyl protein ligase A activity is cultured in a culture medium, said cell secreting enantiomerically pure R-α-lipoic acid in free form into said culture medium and said enantiomerically pure R-α-lipoic acid being removed from said culture medium.

2. A cell secreting enantiomerically pure R-α-lipoic acid into a culture medium and having an attenuated lipoyl protein ligase A activity, wherein it has, instead of a wild-type lplA gene, an lplA allele which has, in the base pair range 367-465, a base substitution which results in the LplA protein activity being reduced by at least 50%, or having a deletion in the lplA gene.

3. The cell as claimed in claim 2, wherein any LplA protein activity is no longer detectable.

4. The cell as claimed in claim 2, wherein it has an increased lipoic acid synthase activity or an increased lipoyl protein ligase B activity.

5. The cell as claimed in claim 2, wherein it is a microorganism such as, for example, a yeast or bacterial strain.

6. The cell as claimed in claim 5, wherein the bacterial strain is of the family Enterobacteriaceae, preferably the species Escherichia coli.

7. The method as claimed in claim 1, wherein a cell secreting enantiomerically pure R-α-lipoic acid into a culture medium and having an attenuated lipoyl protein ligase A activity, wherein it has, instead of a wild-type lplA gene, an lplA allele which has, in the base pair range 367-465, a base substitution which results in the LplA protein activity being reduced by at least 50%, or having a deletion in the lplA gene is used as the cell which has an attenuated lipoyl protein ligase A activity.

8. The method as claimed in claim 1, wherein the enantiomerically pure R-α-lipoic acid is removed by centrifugation of the cell-containing culture medium and subsequent extraction or preceipitation of the R-α-lipoic acid from the cell-free culture medium.

9. The method as claimed in claim 1, wherein the carbon source used in the culture medium is selected from the group of usable sugars, sugar alcohols or organic acids.

10. The method as claimed in claim 1, wherein fatty acids having a chain length of C2-C8, preferably having a chain length of C6-C8 (hexanoic and octanoic acid, respectively), are added to the culture medium.

11. The method as claimed in claim 9, wherein the carbon source is used in a concentration of 0.1-30 g/l.

12. The method as claimed in claim 1, wherein the cells are incubated within the range of the optimum growth temperature for the particular cells over a period of 16-150 h.


SEQUENCE LISTING (4 sequences)

SEQ ID NO: 1 - DNA, 1017 bp, Escherichia coli
  CDS (1)..(1014)
  ref <301>: AUTHORS: Morris, Timothy W. Reed, Kelynne E. Cronan Jr., John E.
  ref <302>: TITLE: Identification of the Gene Encoding Lipoate-Protein Ligase A of Escherichia coli
  ref <303>: JOURNAL: J. Biol. Chem.
  ref <304>: VOLUME: 269
  ref <305>: ISSUE: 23
  ref <306>: PAGES: 16091-16100
  ref <307>: DATE: 1994
atgtccacattacgcctgctcatctctgactcttacgacccgtggtttaacctggcggtggaagagtgtatttttcgccaaatgcccgccacgcagcgcgttctgtttctctggcgcaatgccgacacggtagtaattggtcgcgcgcagaacccgtggaaagagtgtaatacccggcggatggaagaagataacgtccgcctggcgcggcgcagtagcggtggcggcgcggtgttccacgatctcggcaatacctgctttacctttatggctggcaagccggagtacgataaaactatctccacgtcgattgtgctcaatgcgctgaacgcgctcggcgtcagcgccgaagcgtccggacgtaacgatctggtggtgaaaaccgtcgaaggcgaccgcaaagtctcaggctcggcctatcgcgaaaccaaagatcgcggcttccaccacggcaccttgctactcaatgccgacctcagccgcctggcaaactatctcaatccggataaaaagaaactggcggcgaaaggcattacgtcggtacgttcccgcgtgaccaacctcaccgagctgttgccggggatcacccatgagcaggtttgcgaggccataaccgaggcctttttcgcccattatggcgagcgcgtggaagcggaaatcatctccccgaacaaaacgccagacttgccaaacttcgccgaaacctttgcccgccagagtagctgggaatggaacttcggtcaggctccggcattctcgcatctgctggatgaacgctttacctggggcggcgtggaactgcatttcgacgttgaaaaaggccatatcacccgcgcccaggtgtttaccgacagcctcaaccccgcgccgctggaagccctcgccggacgactgcaaggctgcctgtaccgcgcagatatgctgcaacaggagtgcgaagcgctgttggttgacttcccggaacaggaaaaagagctacgggagttatcggcatggatggcgggggctgtaaggtag

SEQ ID NO: 2 - protein, 338 aa, Escherichia coli
MSTLRLLISDSYDPWFNLAVEECIFRQMPATQRVLFLWRNADTVVIGRAQNPWKECNTRRMEEDNVRLARRSSGGGAVFHDLGNTCFTFMAGKPEYDKTISTSIVLNALNALGVSAEASGRNDLVVKTVEGDRKVSGSAYRETKDRGFHHGTLLLNADLSRLANYLNPDKKKLAAKGITSVRSRVTNLTELLPGITHEQVCEAITEAFFAHYGERVEAEIISPNKTPDLPNFAETFARQSSWEWNFGQAPAFSHLLDERFTWGGVELHFDVEKGHITRAQVFTDSLNPAPLEALAGRLQGCLYRADMLQQECEALLVDFPEQEKELRELSAWMAGAVR

SEQ ID NO: 3 - DNA, 30 bp, Artificial Sequence
  Description of Artificial Sequence: Oligonukleotid lplA-fwd
cgggatccctatctgcgcctgacactcgac

SEQ ID NO: 4 - DNA, 33 bp, Artificial Sequence
  Description of Artificial Sequence: Oligonukleotid lplA-rev
cgggatcctttatctgaaccgccatttgcgctg